(19) United States
(12) Patent Application Publication
(10) Pub. No.: US 2012/0269739 A1
(43) Pub. Date: Oct. 25, 2012

(54) COMPOSITION, USE, AND PRESERVATION METHOD

(75) Inventors: Maria Dalko, Versailles (FR); Geraldine Lerebour, Les Loges En Josas (FR); Sylvie Cupferman, L'Hay Les Roses (FR)

(73) Assignee: L'OREAL, Paris (FR)

(21) Appl. No.: 13/499,521

(22) PCT Filed: Sep. 16, 2010
(86) PCT No.: PCT/FR2010/051921

§ 371 Date: Jul. 9, 2012

Related U.S. Application Data
(60) Provisional application No. 61/248,994, filed on Oct. 6, 2009.

(30) Foreign Application Priority Data
Oct. 1, 2009 (FR) 0956833
Nov. 19, 2009 (FR) 0958172

(51) Int. Cl. A61K 8/35; A61K 8/97; C07C 49/255; A61K 8/92; A61K 47/44; A61K 47/46; A61K 47/12; A61K 47/14; A61Q 1/02; A61Q 1/04; A61K 9/12; A61Q 5/06; A61Q 5/04; A61P 17/14; A61P 17/00; A61Q 19/00; A61Q 11/00; A61Q 9/04; A61Q 19/10; A61Q 5/02; A61Q 5/10; A61K 47/10

(52) U.S. Cl. 424/43; 424/49; 514/772; 424/58; 568/308; 514/784; 514/785; 424/63; 424/64; 424/70.1; 424/70.2; 8/161; 510/158; 510/119; 510/130; 8/405; 510/141

(57) ABSTRACT

The invention relates to a cosmetic, pharmaceutical, dermatological, nutraceutical or oral cosmetic composition having, as main preserving system, a mild preserving system formed from compounds with a broad antimicrobial spectrum, which have only one pKa, the said pKa being greater than or equal to 10, the said mild preserving system being present in a content strictly greater than or equal to 1% by weight relative to the weight of the said composition.

The invention also relates to the use, in a cosmetic, dermatological or pharmaceutical composition, of at least one compound of formula (I), as a preserving agent:

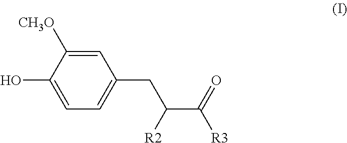

The present invention relates to the use of vanillin derivatives, especially as preserving agents in cosmetic, dermatological, pharmaceutical, nutraceutical or oral cosmetic compositions; and also to cosmetic, pharmaceutical, dermatological, nutraceutical or oral cosmetic compositions having, as main preserving system, a “mild” preserving system.

It is common practice to introduce chemical preserving agents into cosmetic or dermatological compositions, these preserving agents being intended to combat the growth of microorganisms in these compositions, which would quickly make them unsuitable for use. It is in particular necessary to protect compositions against microorganisms capable of growing inside the composition, for example during production thereof, and also against those which the user might introduce therein while handling it, in particular when taking up products in a jar with the fingers.

Chemical preserving agents commonly used are especially parabens, organic acids or formaldehyde-releasing compounds. However, these preserving agents have the drawback of causing irritation, in particular on sensitive skin, when they are present at relatively high levels.

Moreover, in the interests of the environment, consumers are increasingly searching for environmentally friendly, in particular non-ecotoxic, preserving agents.

In addition, the effectiveness of the preserving agents conventionally used is variable and their formulation can pose problems, in particular of incompatibility, or even of destabilization, of formulas, in particular of emulsions.

One object of the present invention is to propose novel preserving agents which in particular have a broad antimicrobial spectrum, at least as broad as, or even broader than, that of the already existing compounds, and which do not have the drawbacks of the prior art, in particular which have specific physicochemical properties making it possible to protect cosmetic formulas against microbial contamination while at the same time being well tolerated.

One subject of the invention is thus a cosmetic, pharmaceutical, dermatological, nutraceutical or oral cosmetic composition having, as main preserving system, a mild preserving system, characterized in that it is formed from a compound with a broad antimicrobial spectrum, which has only one pKa, the said pKa being greater than or equal to 10, or a mixture of such compounds, the said mild preserving system being present in a content strictly greater than 1% and strictly less than 3% by weight relative to the weight of the said composition.

Another subject of the invention is a process for preserving a cosmetic, pharmaceutical, dermatological, nutraceutical or oral cosmetic composition, characterized in that it consists in incorporating into the said composition, as main preserving system, a mild preserving system formed from a compound with a broad antimicrobial spectrum, which has only one pKa, the said pKa being greater than or equal to 10, or a mixture of such compounds, the said mild preserving system being present in a content strictly greater than 1% and strictly less than 3% by weight relative to the weight of the said composition.

Another subject of the invention consists of the use of at least one compound of formula (Ia) as defined below, as a preserving agent, especially in a cosmetic, dermatological, pharmaceutical, nutraceutical or oral cosmetic composition.

Another subject of the invention is a process for preserving a cosmetic, dermatological, pharmaceutical, nutraceutical or oral cosmetic composition, characterized in that it consists in incorporating into the said composition at least one compound of formula (Ia) as defined below.

The mild preserving system is formed exclusively from compounds, alone or as a mixture, as defined in the claim. Other compounds with antimicrobial or preserving properties may be present in the composition, but must be present in total in minor amount; these other compounds are not included in the constitution of the mild preserving system.

The term “as main preserving system” means that the mild preserving system represents at least 50% by weight, or even 60% to 100% by weight and better still 75% to 95% by weight, relative to the total weight of the antimicrobial or preserving compounds present in the composition.

In any case, the mild preserving system according to the invention is present in the composition in a content strictly greater than 1% by weight and strictly less than 3% by weight, relative to the weight of the said composition, especially in a content of between 1.01% and 2.9% by weight, or even between 1.05% and 2.8% by weight, better still between 1.1% and 2% by weight and preferentially between 1.15% and 1.95% by weight.

The compounds present in the mild preserving system according to the invention have a broad antimicrobial spectrum, i.e. they are active on bacteria, yeasts and moulds.

They are especially active on bacteria such as Escherichia coli, Pseudomonas aeruginosa and Enterococcus faecalis, on yeasts such as Candida albicans, and on moulds such as Aspergillus niger.

According to the present invention, the term “preserving agent” means a substance that is commonly added to a composition in order to preserve the said composition with respect to a contaminating agent. Advantageously, the compounds according to the invention are used as antimicrobial and/or antibacterial and/or antifungal agents.

The compounds present in the mild preserving system according to the invention have only one pKa, and this pKa must be greater than or equal to 10.

As a reminder, the pKa corresponds to −log Ka, Ka being the equilibrium constant for the association of the weak acid, or the acidity constant. The pKa may be measured, or calculated or estimated by structural homology with a known compound.

In the case of the compounds of formula (I) according to the invention, the estimated pKa is about 10.27, which is the pKa value for 2-methoxy-4-methylphenol given in the literature. Preferably, the pKa is between 10 and 11.

It is clear that when the mild preserving system according to the invention is formed from several compounds, each of these compounds must have a broad antimicrobial spectrum and only one pKa, and this pKa must be greater than or equal to 10.

Preferably, the mild preserving system according to the invention is formed from compounds, alone or as a mutual mixture, corresponding to formula (I):

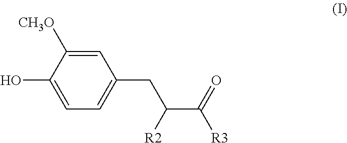

in which:
R2 represents a hydrogen atom or a methyl or ethyl radical;
R3 represents a linear C1-C12 alkyl radical (saturated), optionally substituted with a hydroxyl group; or a linear C2-C12 alkenyl radical (C═C unsaturated), optionally substituted with a hydroxyl group.

A mixture of compounds of formula (I) can of course be used.

Preferably, the composition does not comprise any preserving agents other than those of formula (I). In particular, the composition does not contain parabens.

Preferably, the compounds correspond to formula (I), in which:
R2 is chosen from H and CH₃; better still, R2=H, and/or
R3 represents (i) a C1-C10 alkyl radical; (ii) a C2-C10 alkenyl radical, especially a radical —CH═CH—R4 with R4 representing a linear C1-C6 alkyl radical; or alternatively (iii) a hydroxyalkyl radical of structure —CH₂—CH(OH)—R5 with R5 representing a linear C1-C10 and preferably C4-C10 alkyl radical.

The compounds of formula (I) that are preferred are the following, all these compounds having an estimated pKa of about 10.3:

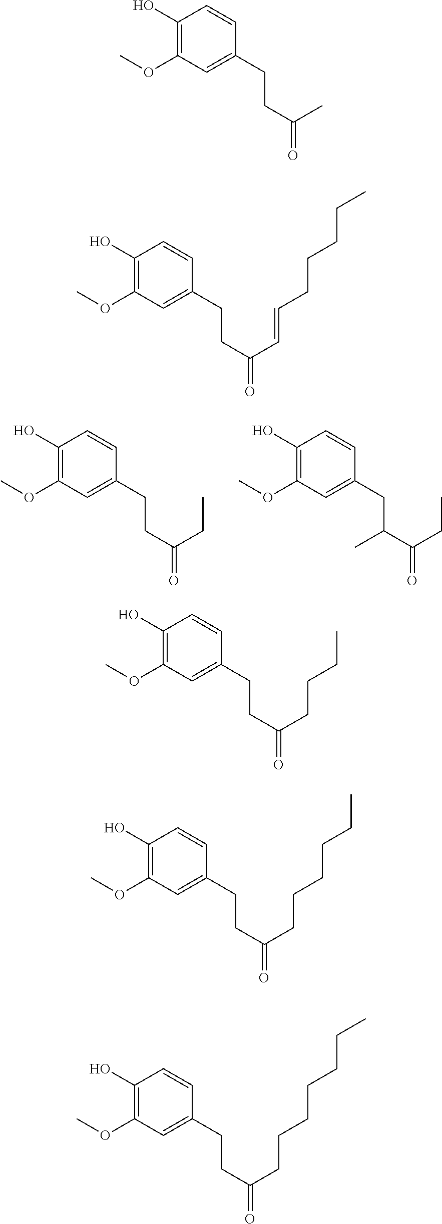

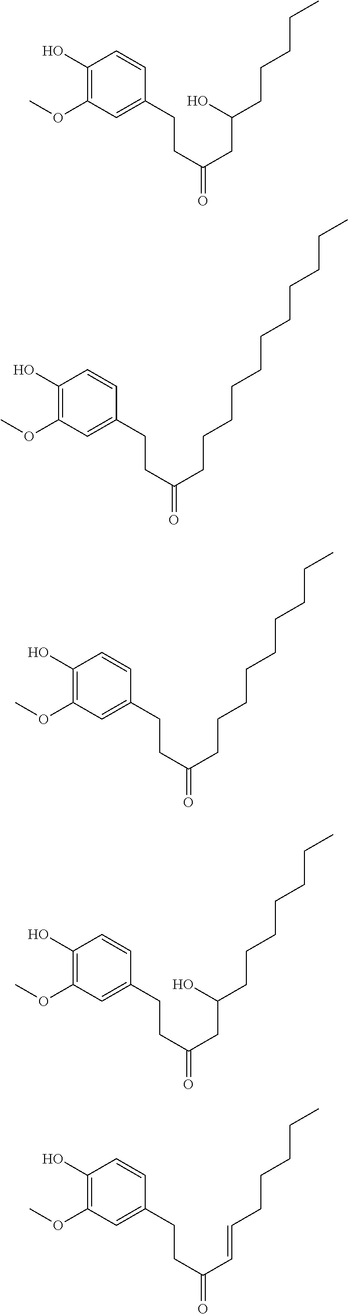

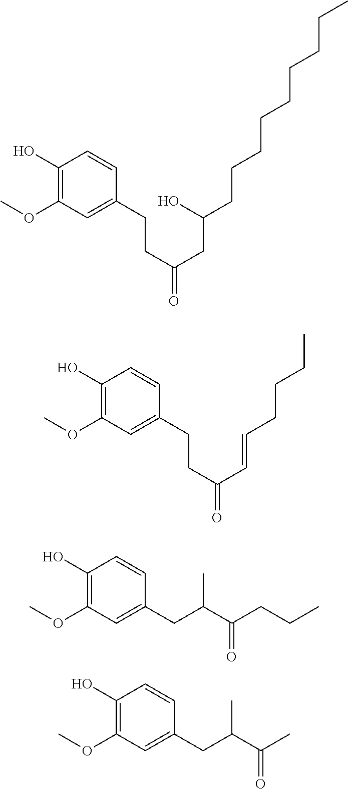

The compounds of formula (I) may be readily prepared by a person skilled in the art on the basis of his general knowledge. Mention may be made especially of the following bibliographic references: J. Asian Natural Products Research, 2006, 8(8), 683-688; Helv. Chimica Acta, 2006, 89(3), 483-495; Chem. Pharm. Bull., 2006, 54(3), 377-379; and Bioorg. J. Med. Chem. Lett., 2004, 14(5), 1287-1289.

They may thus be prepared from vanillin (commercial product) in the following manner:

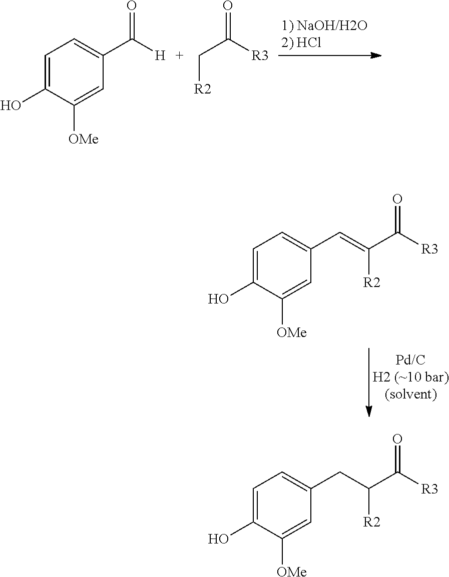

As mentioned hereinabove, the present invention also relates to the use, as a preserving agent, of at least one compound of formula (Ia), especially in a cosmetic, dermatological, pharmaceutical, nutraceutical or oral cosmetic composition:

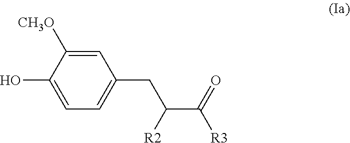

in which:
either R2 represents a hydrogen atom and R3 represents methyl or ethyl,
or R2 represents a methyl or ethyl radical and R3 represents a linear C1-C12 alkyl radical (saturated), optionally substituted with a hydroxyl group; or a linear C2-C12 alkenyl radical (C═C unsaturated), optionally substituted with a hydroxyl group.

A mixture of compounds of formula (Ia) may, of course, be used.

Preferably, the compounds correspond to formula (Ia), in which:
R2=H and R3=methyl or ethyl; or
R2=methyl or ethyl, and R3 represents (i) a C1-C10 and especially C2-C6 alkyl radical; (ii) a C2-C10 alkenyl radical, especially a radical —CH═CH—R4 with R4 representing a linear C1-C6 alkyl radical; or alternatively (iii) a hydroxyalkyl radical of structure —CH₂—CH(OH)—R5 with R5 representing a linear C1-C10 and preferably C4-C10 alkyl radical.

The preferred compounds of formula (Ia) are the following:

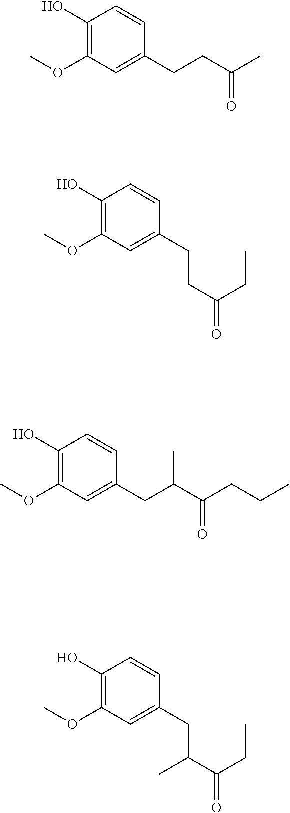

The compounds of formula (Ia) may be present in the composition in a content of greater than or equal to 1% by weight relative to the weight of the said composition, especially between 1% and 5% by weight, or even between 1.5% and 2.5% by weight, and better still between 1.8% and 2.2% by weight.

Certain compounds are novel and as such form another subject of the present invention; these are the compounds of formula (Ib) hereinbelow:

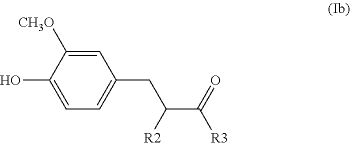

in which:
R2 represents methyl or ethyl, and
R3 represents a linear C1-C12 alkyl radical (saturated), optionally substituted with a hydroxyl group; or a linear C2-C12 alkenyl radical (C═C unsaturated), optionally substituted with a hydroxyl group;
   with the exclusion of the compound in which R2=R3=methyl.

Preferably, the compounds correspond to formula (Ib), in which R2=CH₃.

Preferably, the compounds correspond to formula (Ib), in which R3 represents (i) a C2-C10 and especially C2-C6 alkyl radical; (ii) a C2-C10 alkenyl radical, especially a radical —CH═CH—R4 with R4 representing a linear C1-C6 alkyl radical; or alternatively (iii) a hydroxyalkyl radical of structure —CH₂—CH(OH)—R5 with R5 representing a linear C1-C10 and preferably C4-C10 alkyl radical.

The preferred compounds of formula (Ib) are the following:

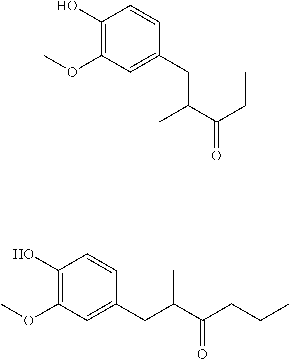

The cosmetic, dermatological or pharmaceutical compositions moreover comprise a cosmetically, dermatologically or physiologically acceptable medium, i.e. a medium that is compatible with keratin materials such as facial or bodily skin, the lips, the hair, the eyelashes, the eyebrows and the nails.

The compositions according to the invention may be in any galenical form conventionally used, in particular for topical application, and in particular in the form of aqueous or aqueous/alcoholic solutions, of oil-in-water (O/W), water-in-oil (W/O) or multiple (triple: W/O/W or O/W/O) emulsions, of aqueous gels or of dispersions of a fatty phase in an aqueous phase using spherules, it being possible for these spherules to be polymeric nanoparticles, such as nanospheres and nanocapsules, or lipid vesicles of ionic and/or nonionic type (liposomes, niosomes or oleosomes), of nanoemulsions, or of thin films. These compositions are prepared according to the usual methods.

The compositions according to the invention may be more or less fluid and may have the appearance of a white or coloured cream, an ointment, a milk, a lotion, a serum, a paste or a foam. They can optionally be applied to the skin in the form of an aerosol. They can also be in solid form, for example in the form of a stick.

The composition according to the invention may in particular be in the form of:
a product for making up the skin of the face, body or lips;
an aftershave gel or lotion;
a hair-removing cream;
a body hygiene composition such as a shower gel or a shampoo;
a pharmaceutical composition;
a solid composition such as a soap or a cleansing bar;
an aerosol composition also comprising a pressurized propellent;
a hairsetting lotion, a hair-styling cream or gel, a dyeing composition, a hair-restructuring lotion, a permanent-waving composition, a lotion or a gel for combating hair loss; or
a composition for oro-dental use.

The physiologically acceptable medium in which the compounds can be used, and also its constituents, their amount, the galenical form of the composition and the method for preparing said composition may be chosen by those skilled in the art on the basis of their general knowledge as a function of the type of composition desired.

In particular, the composition may comprise any fatty substance normally used in the field of application envisaged. Mention may in particular be made of silicone fatty substances such as silicone oils, gums and waxes and also nonsilicone fatty substances such as oils, pastes and waxes of plant, mineral, animal and/or synthetic origin. The oils may be volatile or non-volatile.

Among the silicone oils, mention may be made of volatile or non-volatile polydimethylsiloxanes (PDMSs) with a linear or cyclic silicone chain, which are liquid or pasty at room temperature, in particular cyclopolydimethylsiloxanes such as cyclo-hexasiloxane; polydimethylsiloxanes comprising alkyl, alkoxy or phenyl groups, which are pendent or at the end of a silicone chain, these groups containing from 2 to 24 carbon atoms; phenylsilicones, for instance phenyl trimethicones, phenyl dimethicones, phenyltrimethylsiloxydiphenylsiloxanes, diphenyl dimethicones, di-phenylmethyldiphenyltrisiloxanes or 2-phenylethyl trimethylsiloxy silicates, and polymethylphenylsiloxanes.

Among the hydrocarbon-based oils of plant origin, mention may be made of liquid triglycerides of fatty acids containing from 4 to 10 carbon atoms, for instance heptanoic or octanoic acid triglycerides, or alternatively, for example, sunflower oil, maize oil, soybean oil, marrow oil, grapeseed oil, sesame seed oil, hazelnut oil, apricot oil, macadamia oil, arara oil, sunflower oil, castor oil, avocado oil, caprylic/capric acid triglycerides, jojoba oil and shea butter oil.

Mention may also be made, as a fatty substance that can be used, of
fatty acids containing from 8 to 32 carbon atoms;
synthetic esters and ethers, in particular of formulae R¹COOR² and R¹OR² in which R¹ represents a fatty acid residue containing from 8 to 29 carbon atoms and R² represents a branched or unbranched hydrocarbon-based chain containing from 3 to 30 carbon atoms, for instance purcellin oil, isononyl isononanoate, iso-propyl myristate, 2-ethylhexyl palmitate, 2-octyldodecyl stearate, 2-octyldodecyl erucate or isostearyl isostearate; hydroxylated esters, for instance isostearyl lactate, octyl hydroxystearate, octyldodecyl hydroxystearate, diisostearyl malate, triisocetyl citrate, fatty alcohol heptanoates, octanoates and decanoates; polyol esters, for instance propylene glycol dioctanoate, neopentyl glycol diheptanoate and diethylene glycol diisononanoate; and pentaerythritol esters, for instance pentaerythrityl tetraisostearate;
linear or branched hydrocarbons of mineral or synthetic origin, such as volatile or non-volatile liquid paraffins, and derivatives thereof, petroleum jelly, polydecenes, and hydrogenated polyisobutene such as Parleam oil;
fatty alcohols containing from 8 to 26 carbon atoms, for instance cetyl alcohol, stearyl alcohol and a mixture thereof (cetylstearyl alcohol), octyldodecanol, 2-butyloctanol, 2-hexyldecanol, 2-undecylpentadecanol, oleyl alcohol or linoleyl alcohol.

The composition may also comprise an aqueous medium that comprises water, an aqueous-alcoholic medium containing a C2-C6 alcohol such as ethanol or isopropanol, or an organic medium comprising standard organic solvents such as C2-C6 alcohols, in particular ethanol and isopropanol, glycols such as propylene glycol, and ketones.

The composition according to the invention may also comprise the adjuvants that are customary in the cosmetic and dermatological fields, such as thickeners, emulsifiers, surfactants, gelling agents, active cosmetic agents, fragrances, fillers, dyestuffs, moisturizers, vitamins and polymers. The amounts of these various adjuvants are those conventionally used in the fields under consideration, for example from 0.001% to 20% of the total weight of the composition. These adjuvants and the concentrations thereof must be such that they are not detrimental to the advantageous properties of the compounds according to the invention.

The pH of the compositions according to the invention, when they comprise at least one aqueous phase (for example aqueous solutions, emulsions), is preferably between 4 and 9, preferably between 4 and 7, advantageously between 5 and 6.

The invention is illustrated in greater detail in the following exemplary embodiments.

### EXAMPLE 1

The antimicrobial efficacy of a compound of formula (I) was evaluated via the Challenge Test or artificial contamination method.

### Compound Tested:

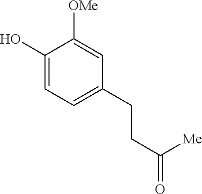

### Protocol

The method of the challenge test consists of an artificial contamination of the sample with collection microbial strains (bacteria, yeasts and moulds) and evaluation of the number of revivable microorganisms seven days after the inoculation.

In order to demonstrate the efficacy of the compounds of formula (I) the antimicrobial activity of a cosmetic formula containing a compound according to the invention was compared with the same formula alone (control), after inoculation with about 10⁶ cfu (colony-forming units)/gram of cosmetic formula.

### Microorganism Cultures

5 pure microorganism cultures are used.

| MICROORGANISMS | Subculturing medium | T° | ATCC |
|---|---|---|---|
| Escherichia coli (Ec) | Trypto-casein soya | 35° C. | 8739 |
| Enterococcus faecalis (Ef) | Trypto-casein soya | 35° C. | 33186 |
| Pseudomonas aeruginosa (Pa) | Trypto-casein soya | 35° C. | 19429 |
| Candida albicans (Ca) | Sabouraud | 35° C. | 10231 |
| Aspergillus niger (An) | Malt | 35° C. | 6275 |

ATCC = American Type Culture Collection

The gram-negative bacteria (Escherichia coli and Pseudomonas aeruginosa), gram-positive bacteria (Enterococcus faecalis), yeast (Candida albicans) and mould (Aspergillus niger) are seeded in the subculturing medium, respectively, the day before the inoculation for the bacteria and the yeast, and five days before the inoculation for the mould.

On the day of inoculation:
a suspension in Tryptone salt diluent is prepared, respectively, for the bacteria and the yeast, so as to obtain in a spectrophotometer a suspension with an optical density of between 35% and 45% transmitted light at 544 nm;
for the mould, the spores are collected by washing the agar with 6 to 7 ml of harvesting solution and the suspension is recovered in a sterile tube or flask.

After homogenizing the microbial suspension, 0.2 ml of inoculum is introduced into each pill bottle (the suspensions are used pure: between 1×10⁸ and 3×10⁸ cfu per ml) and the microbial suspension in the 20 g of product (=cosmetic formula) is homogenized thoroughly using a spatula.

The content of microorganisms present in the product corresponds after homogenization to a concentration of 10⁶ microorganisms per gram of product, i.e. inoculation to 1% of an inoculum containing 10⁸ microorganisms per ml.

After 7 days of contact time between the microorganisms and the product at 22° C.±2° C. and in darkness, decimal dilutions are performed and the number of revivable microorganisms remaining in the product is counted.

### 1st Formula Tested: O/W Emulsion (Weight %)

| | |
|---|---|
| sodium hydroxide | 0.03% |
| liquid petroleum jelly | 10% |
| 2-ethylhexyl palmitate | 10% |
| acrylic acid/stearyl methacrylate copolymer | 0.1% |
| polymerized in an ethyl acetate/cyclohexane mixture | |
| glycerol | 5% |
| mixture of cetylstearyl glucoside and of cetyl | 2.45% |
| and stearyl alcohols (12/46/42) | |
| microbiologically clean deionized water | qs 100% |

Antimicrobial activity of the compound according to the invention, in comparison with that of conventional preserving agents, expressed as the Log reduction number after 7 days of contact, relative to the control:

| | | | P. aeru- | E. fae- | C. albi- | A. |
|---|---|---|---|---|---|---|
| | Amt* | E. coli | ginosa | calis | cans | niger |
| Phenoxyethanol | 1% | >4 | >4 | 0.6 | 2.5 | 3.3 |
| Sodium benzoate | 0.6% | 0.3 | 0.6 | 0.6 | 0 | 0.5 |
| Chlorhexidine | 0.3% | >4 | 0.3 | 0.9 | 0.3 | 0.4 |
| digluconate | | | | | | |
| Gingerone | 2% | >4 | >4 | >4 | >4 | >4 |

*Amt: amount of compound tested, in the composition (weight %)

### 2nd Formula Tested: Lotion

| | |
|---|---|
| allantoin | 0.05% |
| sodium chloride | 0.09% |
| citric acid | qs pH 7 ± 0.2 |
| cornflower water | 1% |
| hexylene glycol (2-methyl-2,4-pentanediol) | 1% |
| glycerol | 5% |
| sodium N-cocoylamidoethyl-N-ethoxycarboxymethyl | 1.1% |
| glycinate | |
| sodium/magnesium lauryl ether sulfate (80/20) | 0.45% |
| 40 OE (52% SM) | |
| microbiologically clean deionized water | qs 100% |

### Results:

| | | | P. aeru- | E. fae- | Candida | A. |
|---|---|---|---|---|---|---|
| | Amt* | E. coli | ginosa | calis | albicans | niger |
| Sodium benzoate | 0.6% | 0.7 | <0.5 | 1.4 | <0.5 | <0.5 |
| Phenoxyethanol | 1% | >4 | >4 | >3 | >4 | 1.5 |
| Chlorhexidine | 0.3% | 0.8 | 0.5 | 1.3 | 3 | 0.4 |
| digluconate | | | | | | |
| Gingerone | 2% | >4 | >3 | >0.5 | >3 | >4 |

*Amt: amount of compound tested, in the composition (weight %)

### 3rd Formula Tested: O/W Emulsion

| | |
|---|---|
| sorbitan tristearate (Span 65 V ® from Croda) | 0.9% |
| polyethylene glycol stearate (40 OE) | 2.0% |
| (Myrj 52 P ® from Croda) | |
| glyceryl mono-distearate (36/64)/potassium | 3.0% |
| stearate mixture | |
| fatty acids of plant origin (53/44/3 stearic | 1.2% |
| acid/palmitic acid/myristic acid) | |
| glycerol | 3.0% |
| hydrogenated isoparaffin | 7.2% |
| cyclopentasiloxane | 5.0% |
| white petroleum jelly | 4.0% |
| cetyl alcohol | 4.0% |
| sodium hydroxide | 0.05% |
| caffeine | 0.1% |
| citric acid | 0.04% |
| liquid fraction of shea butter | 1% |
| apricot kernel oil | 0.3% |
| tetradecanoic acid, tetradecyl ester | 2% |
| water | qs 100% |

### Results:

| | | | P. aeru- | E. fae- | Candida | A. |
|---|---|---|---|---|---|---|
| | Amt* | E. coli | ginosa | calis | albicans | niger |
| Sodium benzoate | 0.6% | >4 | >4 | >4 | >4 | 3 |
| Chlorhexidine | 0.3% | >4 | 0 | 1.8 | >4 | 2.3 |
| digluconate | | | | | | |
| Gingerone | 2% | >4 | >4 | >4 | >4 | 0.6 |

It is found that, in contrast with conventional preserving agents whose activity varies as a function of the microbial strain and of the formulatory support, the compound tested (gingerone) is efficient irrespective of the strain and of the support.

### EXAMPLE 2

The object of this example is to distinguish a compound according to the invention (gingerone) from the preserving agents conventionally used for protecting cosmetic formulations against microbial contamination, as regards the action kinetics.

### Solutions Tested:

The solutions are prepared in Trypto-casein-soya (TS) broth for the bacteria and in Sabouraud broth for the yeast. These solutions are adjusted to pH 5.2±0.3.

| | Test concentration (weight %) in |
|---|---|
| Molecule | the final mixture |
| Gingerone | 2% |
| Sodium benzoate | 3.3% (i.e. 2.5% acid) |
| Chlorhexidine digluconate | 2.2% (i.e. 0.3% base chlorhexidine) |

For each microbial solution, a growth control consisting of the appropriate culture medium alone is prepared.

### Strains Tested:

| Bacteria | Yeast |
|---|---|
| Staphylococcus aureus (ATCC 6538) | Candida albicans (ATCC 10231) |
| Enterococcus faecalis (ATCC 33186) | |

### Procedure

The solutions in TS broth (36 ml) are inoculated separately with each of the bacteria (4 ml of microbial suspension) to a final content of 10⁶ cfu/ml of mixture.

The solutions in Sabouraud broth (36 ml) are inoculated with the yeast (4 ml of suspension) to a final content of 10⁶ cfu/ml.

The inoculated samples are then placed in an incubator-agitator at 35° C.±2° C. Samples are taken after 6 hours, 24 hours, 48 hours, 72 hours and 7 days. Decimal dilutions are then prepared in Eugon LT100 broth. These dilutions are left for 30 minutes±15 minutes at 25° C. in order to neutralize the preserving agents, and are then double-seeded on the surface of Petri dishes containing Eugon LT100 agar.

The Petri dishes are then incubated in an oven at 35° C.±2° C. for a variable time depending on the strain concerned:
48 hours; S. aureus;
5 days: E. faecalis and C. albicans.

### Results

The results are expressed for each solution, each strain and each contact time as the number of cfu/ml of stock solution.

TABLE 1
Action kinetics with respect to Staphylococcus aureus

| | | Number of cfu counted after the various | | | | | |
|---|---|---|---|---|---|---|---|
| | | strain/solution contact times (in hours) | | | | | |
| | * | 0 | 6 | 24 | 48 | 72 | 168 |
| Growth | | 1.7 × 10⁶ | 4.3 × 10⁷ | 5.4 × 10⁶ | 3.1 × 10⁵ | 3.3 × 10⁵ | 1.4 × 10⁷ |
| control | | | | | | | |
| Gingerone | 2% | 1.7 × 10⁶ | 1.2 × 10⁶ | 1.7 × 10⁵ | 8.1 × 10³ | 40 | <100 |
| Sodium | 3.3% | 1.7 × 10⁶ | <10 | <10 | <10 | <10 | <10 |
| benzoate | | | | | | | |
| Chlorhexi- | 2.2% | 1.7 × 10⁶ | <10 | <10 | <10 | <10 | <10 |
| dine diglu- | | | | | | | |
| conate | | | | | | | |
| Growth | | 2.5 × 10⁶ | 4.4 × 10⁷ | 4.1 × 10⁶ | 2.9 × 10⁵ | 4.6 × 10⁵ | 1.5 × 10⁷ |
| control | | | | | | | |
| Gingerone | 2% | 2.5 × 10⁶ | 1.2 × 10⁶ | 1.6 × 10⁵ | 9.6 × 10³ | 1.3 × 10² | <10 |
| Sodium | 3.3% | 2.5 × 10⁶ | <10 | <10 | <10 | <10 | <10 |
| benzoate | | | | | | | |
| Chlorhexi- | 2.2% | 2.5 × 10⁶ | <10 | <10 | <10 | <10 | <10 |
| dine diglu- | | | | | | | |
| conate | | | | | | | |

* test concentration

TABLE 2
Action kinetics with respect to Enterococcus faecalis

| | | Number of cfu counted after the various | | | | | |
|---|---|---|---|---|---|---|---|
| | | strain/solution contact times (in hours) | | | | | |
| | * | 0 | 6 | 24 | 48 | 72 | 168 |
| Growth | | 2.5 × 10⁶ | 2.5 × 10⁸ | 4.5 × 10⁸ | 2.5 × 10⁸ | 1.7 × 10⁸ | 1.9 × 10⁷ |
| control | | | | | | | |
| Gingerone | 2% | 2.5 × 10⁶ | 4.4 × 10⁵ | 1.2 × 10⁴ | 6.3 × 10² | 80 | <10 |
| Sodium | 3.3% | 2.5 × 10⁶ | 4.4 × 10⁵ | 7.0 × 10² | <10 | <10 | <10 |
| benzoate | | | | | | | |
| Chlorhexi- | 2.2% | 2.5 × 10⁶ | <10 | <10 | <10 | <10 | <10 |
| dine diglu- | | | | | | | |
| conate | | | | | | | |

* test concentration

TABLE 3
Action kinetics with respect to Candida albicans

| | | Number of cfu counted after the various | | | | | |
|---|---|---|---|---|---|---|---|
| | | strain/solution contact times (in hours) | | | | | |
| | * | 0 | 6 | 24 | 48 | 72 | 168 |
| Growth | | 2.9 × 10⁶ | 2.3 × 10⁷ | 5.2 × 10⁷ | 2.3 × 10⁷ | 1.9 × 10⁷ | 2.9 × 10⁶ |
| control | | | | | | | |
| Gingerone | 2% | 2.9 × 10⁶ | 2.9 × 10² | <10 | <10 | <10 | <10 |
| Sodium | 3.3% | 2.9 × 10⁶ | <10 | <10 | <10 | <10 | <10 |
| benzoate | | | | | | | |
| Chlorhexi- | 2.2% | 2.9 × 10⁶ | <10 | <10 | <10 | <10 | <10 |
| dine diglu- | | | | | | | |
| conate | | | | | | | |

* test concentration

### Conclusion

The action kinetics of the conventional cosmetic preserving agents, sodium benzoate and chlorhexidine digluconate, are rapid (generally less than 6 hours). In comparison, the activity of gingerone is expressed much more slowly, in particular on the bacteria.

The antimicrobial activity of the family of molecules according to the invention is significantly different from that of the closest conventional cosmetic preserving agents, both as regards the physicochemical properties (high pKa, greater than 10: chlorhexidine pKa=10.3), and as regards the working concentration (>1%).

### EXAMPLE 3

### Lotion

A lotion is prepared, comprising (weight %):

| | |
|---|---|
| compound tested in Example 1 (gingerone) | 0.75% |
| glycerol | 2% |
| ethyl alcohol | 20% |
| oxyethylenated (26 OE) and oxypropylenated (26 OP) | 1% |
| butanol, oxyethylenated (40 OE) hydrogenated castor | |
| oil in water | |
| demineralized water | qs 100% |

### EXAMPLE 4

### Facial Gel

A facial gel is prepared, comprising (weight %):

| | |
|---|---|
| polyglyceryl acrylate (Norgel) | 30% |
| polyacrylamide/C13-14 isoparaffin/laureth-7 (Sepigel 305) | 2% |
| silicone oil | 10% |
| compound 1 (gingerone) | 5% |
| water | qs 100% |

### EXAMPLE 5

### Treating Gel

A treating gel is prepared, comprising (weight %):

| | |
|---|---|
| compound 1 (gingerone) | 1% |
| xanthan gum | 1% |
| glycerol | 2% |
| ethanol | 20% |
| mixture of oxyethylenated (26 OE) and oxypropylenated | 1% |
| (26 OP) butyl alcohol, oxyethylenated (40 OE) hydro- | |
| genated castor oil in water | |
| fragrance | qs |
| demineralized water | qs 100% |

### EXAMPLE 6

### Foaming Cleansing Cream

A foaming cleansing cream is prepared, comprising (weight %):

| | |
|---|---|
| ethylene glycol monostearate | 2% |
| compound 1 (gingerone) | 0.5% |
| magnesium aluminium silicate hydrate | 1.7% |
| hydroxypropylmethylcellulose | 0.8% |
| mixture of sodium cocoyl isethionates and coconut | 15% |
| fatty acids (Elfan AT 84 G from Akzo) | |
| stearic acid | 1.25% |
| sodium lauroyl sarcosinate at 30% in water | 10% |
| fragrance | qs |
| demineralized water | qs 100% |

### EXAMPLE 7

### Care Cream

A care cream is prepared, comprising (weight %):

| | |
|---|---|
| sorbitan tristearate | 1% |
| compound 1 (gingerone) | 1.5% |
| crosslinked carboxyvinyl homopolymer | 0.4% |
| xanthan gum | 0.5% |
| ethylene glycol dimethacrylate/ | 1% |
| lauryl methacrylate copolymer | |
| cyclopentadimethylsiloxane | 6% |
| glycerol | 3% |
| emulsifier | 4% |
| fragrance | qs |
| demineralized water | qs 100% |

### EXAMPLE 8

### Cover Stick

A cover stick is prepared, comprising (weight %):

| | |
|---|---|
| waxes (carnauba wax and ozokerite) | 14% |
| liquid fraction of shea butter | 4% |
| titanium and zinc oxides | 22% |
| iron oxides | 4% |
| compound 1 (gingerone) | 1% |
| polydimethylsiloxane/hydrated silica | 0.1% |
| cetyl alcohol | 1.4% |
| isoparaffin | qs 100% |

### EXAMPLE 9

### Tinted Cream

A tinted cream is prepared, comprising (weight %):

| | |
|---|---|
| hydrogenated lecithin | 2.4% |
| apricot kernel oil | 6% |
| ethylene glycol dimethacrylate/ | 1% |
| lauryl methacrylate copolymer | |
| oxyethylenated (5 OE) soya sterols | 1.6% |
| compound 1 (gingerone) | 1% |
| iron oxides | 0.9% |
| titanium oxide | 5% |
| polyacrylamide/C13-14 isoparaffin/laureth-7 | 4% |
| (Sepigel 305) | |
| cyclopentadimethylsiloxane | 6% |
| glycerol | 6% |
| propylene glycol | 6% |
| fragrance | qs |
| demineralized water | qs 100% |

1. Cosmetic, pharmaceutical, dermatological, nutraceutical or oral cosmetic composition having, as main preserving system, a mild preserving system, wherein it is formed from a comTraitement canitie avec Shou Wu +anti-chute (ER4017)pound with a broad antimicrobial spectrum, which has only one pKa, the said pKa being greater than or equal to 10, or a mixture of such compounds, the said mild preserving system being present in a content strictly greater than 1% and strictly less than 3% by weight relative to the weight of the said composition.

2. Composition according to claim 1, in which the mild preserving system represents at least 50% by weight, or even 60% to 100% by weight and better still 75% to 95% by weight, relative to the total weight of the antimicrobial or preserving compounds present in the composition.

3. Composition according to claim 1, in which the mild pre-serving system is present in the composition in a content of between 1.01% and 2.9% by weight, or even between 1.05% and 2.8% by weight, better still between 1.1% and 2% by weight and preferentially between 1.15% and 1.95% by weight relative to the weight of the said composition.

4. Composition according to claim 1, in which the mild pre-serving system is formed from compounds, alone or as a mutual mixture, corresponding to formula (I): 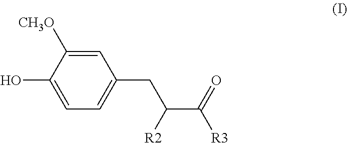
in which:
R2 represents a hydrogen atom or a methyl or ethyl radical;
R3 represents a linear C1-C12 alkyl radical (saturated), optionally substituted with a hydroxyl group; or a linear C2-C12 alkenyl radical (C═C unsaturated), op-tionally substituted with a hydroxyl group.

5. Composition according to claim 4, in which, in formula (I):
R2 is chosen from H and CH₃; better still, R2=H, and/or
R3 represents (i) a C1-C10 alkyl radical; (ii) a C2-C10 alkenyl radical, especially a radical —CH═CH—R4 with R4 representing a linear C1-C6 alkyl radical; or alterna-tively (iii) a hydroxyalkyl radical of structure —CH2-CH(OH)—R5 with R5 representing, a linear C1-C10 and preferably C4-C10 alkyl radical.

6. Composition according to claim 1, in which the mild pre-serving system is formed from compounds, alone or as a mutual mixture, chosen from:

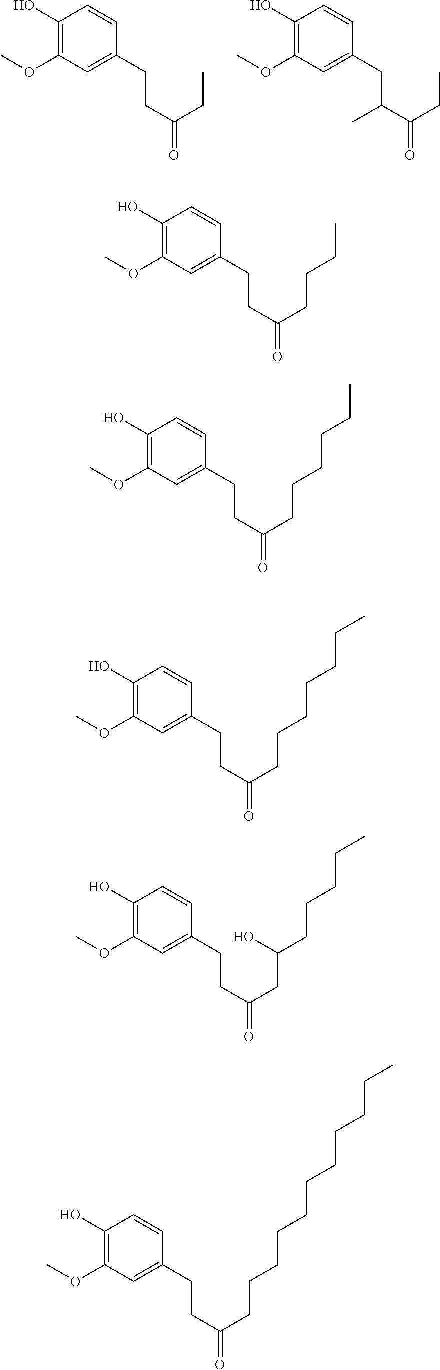

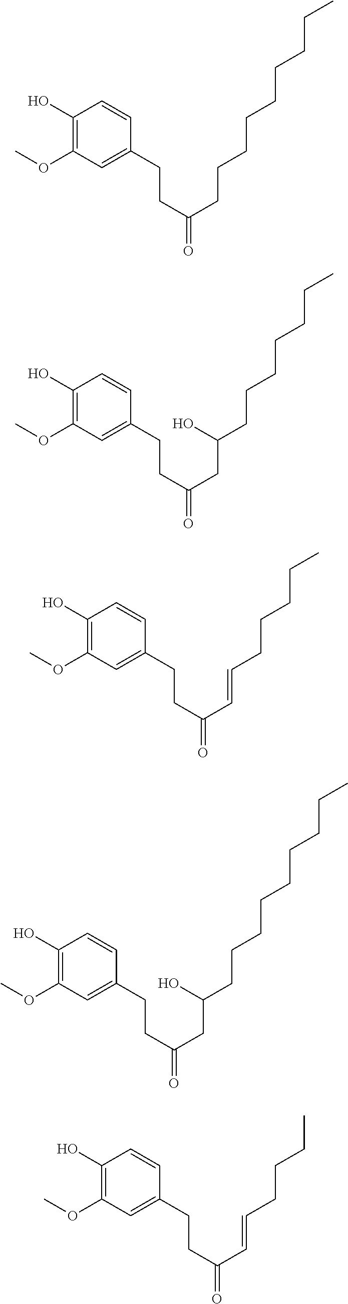

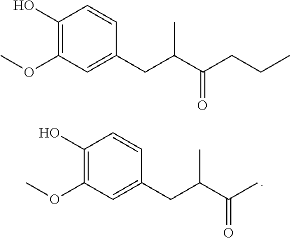

7. Composition according to claim 1, comprising a physiologi-cally acceptable medium which comprises at least one ingredient chosen from silicone fatty substances such as silicone oils, gums and waxes; non-silicone fatty substances such as oils, pastes and waxes of plant, mineral, animal and/or synthetic origin; fatty acids having from 8 to 32 carbon atoms; synthetic esters and ethers, in particular of formula R1COOR2 and R1OR2 in which R1 represents the residue of a fatty acid comprising from 8 to 29 carbon atoms, and R2 represents a branched or unbranched hydrocarbon-based chain containing from 3 to 30 carbon atoms; linear or branched hydrocarbons of mineral or synthetic origin; fatty alcohols having from 8 to 26 carbon atoms; water; C2-C6 alcohols; glycols such as propylene glycol, ketones; thickeners, emulsifiers, surfactants, gelling agents, active cosmetic agents, fragrances, fillers, colorants, moisturizers, vitamins and polymers.

8. Composition according to claim 1, which is in the form of a product for making up the skin of the face, body or lips; an aftershave gel or lotion; a hair-removing cream; a body hygiene composition such as a shower gel or a shampoo; a pharmaceutical composition; a solid composition such as a soap or a cleansing bar; an aerosol composition also comprising a pressurized propellent; a hairsetting lotion, a hair-styling cream or gel, a dyeing composition, a hair-restructuring lotion, a permanent-waving composition, a lotion or a gel for combating hair loss; or a composition for oro-dental use.

9. Use of at least one compound of formula (Ia) as a preserving agent, especially in a cosmetic, dermatological, pharmaceutical, nutraceutical or oral cosmetic com-position: 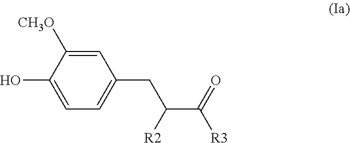
in which:
either R2 represents a hydrogen atom and R3 represents methyl or ethyl,
or R2 represents a methyl or ethyl radical and R3 represents a linear C1-C12 alkyl radical (saturated), optionally substituted with a hydroxyl group; or a linear C2-C12 alkenyl radical (C═C unsaturated), optionally substituted with a hydroxyl group.

10. Use according to claim 9, in which, in formula (Ia):
R2=H and R3=methyl or ethyl; or
R2=methyl or ethyl, and R3 represents (i) a C1-C10 and especially C2-C6 alkyl radical; (ii) a C2-C10 alkenyl radical, especially a radical —CH═CH—R4 with R4 rep-resenting a linear C1-C6 alkyl radical; or alternatively (iii) a hydroxyalkyl radical of structure —CH₂—CH(OH)—R5 with R5 representing a linear C1-C10 and preferably C4-C10 alkyl radical.

11. Use according to claim 9, in which the compound of formula (Ia) is chosen, alone or as a mixture, from the following compounds:

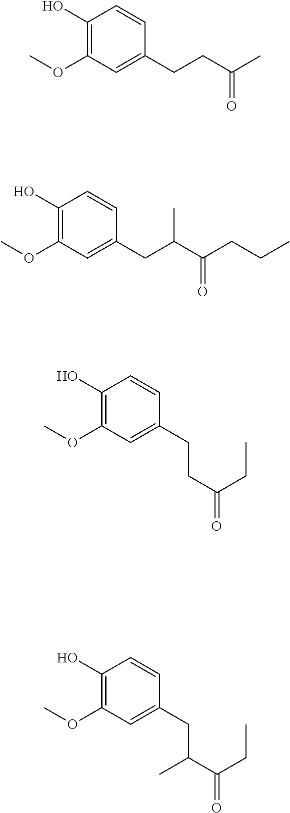

12. Use according to claim 9, in which the composition comprises a physiologically acceptable medium which comprises at least one ingredient cho-sen from silicone fatty substances such as silicone oils, gums and waxes; non-silicone fatty substances such as oils, pastes and waxes of plant, mineral, animal and/or synthetic origin; fatty acids having from 8 to 32 carbon atoms; synthetic esters and ethers, in particular of formula R1COOR2 and R10R2 in which R1 represents the residue of a fatty acid comprising from 8 to 29 carbon atoms, and R2 represents a branched or unbranched hydrocarbon-based chain containing from 3 to 30 carbon atoms; linear or branched hydrocarbons of mineral or synthetic origin; fatty alcohols having from 8 to 26 carbon atoms; water; C2-C6 alcohols; glycols such as propylene glycol, ketones; thickeners, emulsifiers, surfactants, gelling agents, active cosmetic agents, fragrances, fillers, colorants, moisturizers, vitamins and polymers.

13. Use according to claim 9, in which the composition is in the form of a product for making up the skin of the face, body or lips; an aftershave gel or lotion; a hair-removing cream; a body hygiene composition such as a shower gel or a shampoo; a pharmaceutical composition; a solid composition such as a soap or a cleansing bar; an aerosol composition also comprising a pressurized propellent; a hairsetting lotion, a hair-styling cream or gel, a dyeing composition, a hair-restructuring lotion, a permanent-waving composition, a lotion or a gel for combating hair loss; a composition for oro-dental use.

14. Process for preserving a cosmetic, dermatological, pharmaceutical, nutraceuti-cal or oral cosmetic composition, wherein it consists in incorporating into the said composition at least one compound of formula (Ia) as defined in one of claim 9.

15. Process for preserving a cosmetic, pharmaceutical, dermatological, nutraceuti-cal or oral cosmetic composition, wherein it consists in incorporating into the said composition, as main preserving system, a mild preserving system formed from a compound with a broad antimicrobial spectrum, which has only one pKa, the said pKa being greater than or equal to 10, or a mixture of such corn-pounds, the said mild preserving system being present in a content strictly greater than or equal to 1% by weight and strictly less than 3% by weight relative to the weight of the said composition.

16. Compound of formula (Ib): 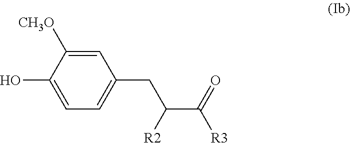
in which:
R2 represents methyl or ethyl, and
R3 represents a linear C1-C12 alkyl radical (saturated), optionally substituted with a hydroxyl group; or a linear C2-C12 alkenyl radical (C═C unsaturated), op-tionally substituted with a hydroxyl group;
with the exclusion of the compound in which R2=R3=methyl.

17. Composition according claim, in which the mild pre-serving system is present in the composition in a content of between 1.01% and 2.9% by weight, or even between 1.05% and 2.8% by weight, better still between 1.1% and 2% by weight and preferentially between 1.15% and 1.95% by weight relative to the weight of the said composition.

18. Composition according to claim 2, in which the mild pre-serving system is formed from compounds, alone or as a mutual mixture, corre-sponding to formula (I): 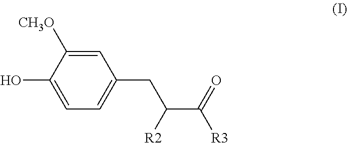
in which:
R2 represents a hydrogen atom or a methyl or ethyl radical;
R3 represents a linear C1-C12 alkyl radical (saturated), optionally substituted with a hydroxyl group; or a linear C2-C12 alkenyl radical (C═C unsaturated), op-tionally substituted with a hydroxyl group.

19. Composition according to claim 3, in which the mild pre-serving system is formed from compounds, alone or as a mutual mixture, corre-sponding to formula (I): 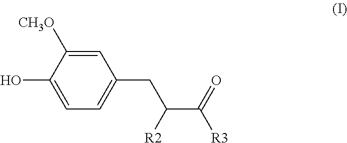
in which:
R2 represents a hydrogen atom or a methyl or ethyl radical;
R3 represents a linear C1-C12 alkyl radical (saturated), optionally substituted with a hydroxyl group; or a linear C2-C12 alkenyl radical (C═C unsaturated), op-tionally substituted with a hydroxyl group.

20. Composition according to claim 2, in which the mild pre-serving system is formed from compounds, alone or as a mutual mixture, chosen from: 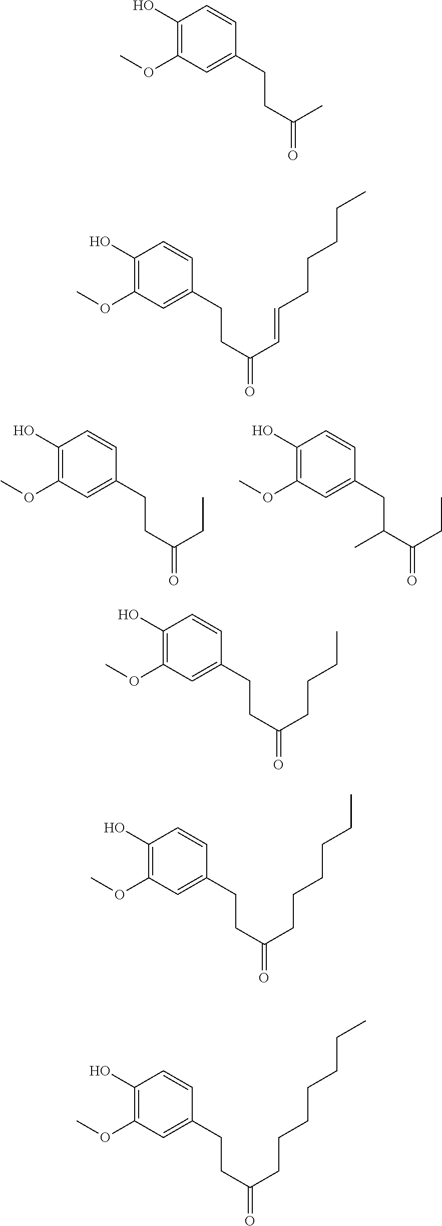 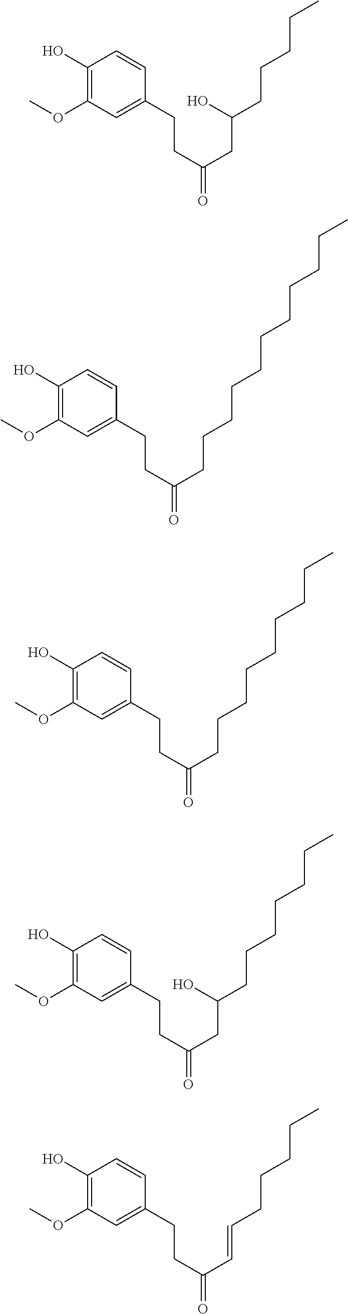 [chemical structure]
